(19) United States
(12) Patent Application Publication
(10) Pub. No.: US 2008/0166373 A1
(43) Pub. Date: Jul. 10, 2008

(54) METHODS AND COMPOSITIONS FOR DELIVERY AND EXPRESSION OF INTERFERON-alpha NUCLEIC ACIDS

(75) Inventors: Tattanahalli L. Nagabhushan, Parsippany, NJ (US); Deba P. Saha, Nutley, NJ (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Canji, Inc., San Diego, CA (US)

(21) Appl. No.: 12/053,479

(22) Filed: Mar. 21, 2008

Related U.S. Application Data
(63) Continuation of application No. 09/353,423, filed on Jul. 15, 1999.
Application No. 09/353,423 is a continuation-in-part of application No. 08/950,927, filed on Oct. 15, 1997, now abandoned.
(60) Provisional application No. 60/028,700, filed on Oct. 18, 1996.

(51) Int. Cl. A61K 39/235; A61P 31/12

(52) U.S. Cl. 424/205.1

(57) ABSTRACT

The present invention is directed to compositions and methods for the delivery of interferon polypeptides. The invention provides recombinant viral and non-viral vectors for the selective expression of interferon polypeptides in particular cell or tissue types. The invention further provides pharmaceutically acceptable formulations of such vectors for administration to mammalian subjects. The invention further provides methods of treatment of diseases in mammalian organisms through the delivery of recombinant vectors selectively expressing interferon polypeptides.

### CROSS-REFERENCE TO RELATED APPLICATIONS

This application is a Continuation of U.S. patent application Ser. No. 09/353,423, filed Jul. 15, 1999, which is a Continuation-In-Part of U.S. patent application Ser. No. 08/950,927, filed Oct. 15, 1997, now abandoned, which claimed priority to U.S. Provisional Patent Application No. 60/028,700 filed Oct. 18, 1996.

### BACKGROUND OF THE INVENTION

The human interferon alphas (IFN-oc) are a family of proteins comprising at least 24 subspecies (Zoon, K.C., Interferon 9:1 (1987), Gresser, I., ed., Academic Press, N.Y.). The interferon αs were originally described as agents capable of inducing an antiviral state in cells but are now known as pleiotropic lymphokines affecting many functions of the immune system (Openakker, et al. Experimentia 45:513 (1989)).

IFN-α has been widely used for therapeutic purposes, including hairy cell leukemia, kaposi's sarcoma, renal cell carcinoma, non Hodgkin's lymphoma, T-cell leukemia, multiple and chronic myelogenous leukemia, malignant melanoma, bladder cell carcinoma, colon carcinoma (with 5-FU), condyloma acuminata, rhinovirus and various forms of chronic viral hepatitis occurring as a result of hepatitis B virus (HBV), hepatitis C virus (HCV), non A non B virus (NANB), or hepatitis δ virus (HDV) infection (Pestka AIDA Research &Human Retroviruses 8(5):776-786 (1992)). IFN-α has also been found to be highly effective against megakaryocytopoiesis and controlling thrombocytosis in patients with myeloproliferative disorders (Talpaz, et al. Annals Int. Med. 99:789-792 (1983); Gisslinger, et al. Lancet:634-637 (1989); Ganser, et al. Blood 70:1173-1179(1987)).

Gene therapy techniques have the potential for limiting the exposure of a subject to a gene product, such as interferon, by targeting the expression of the therapeutic gene to a tissue of interest. However, in general, the ability to target the tissue of interest is one of the major challenges of gene therapy. As an example of the targeting of interferon genes, WIPO Patent Application Publication No. WO 93/15609 discloses the delivery of interferon genes to vascular tissue by administration of such genes to areas of vessel wall injury using a catheter system. In another example, an adenoviral vector encoding a protein capable of enzymatically converting a prodrug, a “suicide gene”, and a gene encoding a cytokine are administered directly into a solid tumor.

Other methods of targeting therapeutic genes to tissues of interest include the three general categories of transductional targeting, positional targeting, and transcriptional targeting (for a review, see, e.g., Miller, et al. FASEB J. 9:190-199 (1995)). Transductional targeting refers to the selective entry into specific cells, achieved primarily by selection of a receptor ligand. Positional targeting within the genome refers to integration into desirable loci, such as active regions of chromatin, or through homologous recombination with an endogenous nucleotide sequence such as a target gene. Transcriptional targeting refers to selective expression attained by the incorporation of transcriptional promoters with highly specific regulation of gene expression tailored to the cells of interest.

Examples of tissue-specific promoters include the promoter for creatine kinase, which has been used to direct the expression of dystrophin cDNA expression in muscle and cardiac tissue (Cox, et al. Nature 364:725-729 (1993)); and immunoglobulin heavy or light chain promoters for the expression of suicide genes in B cells (Maxwell, et al. Cancer Res. 51:4299-4304 (1991)). An endothelial cell-specific regulatory region has also been characterized (Jahroudi, et al. Mol. Cell. Biol. 14:999-1008 (1994)). Amphotrophic retroviral vectors have been constructed carrying a herpes simplex virus thymidine kinase gene under the control of either the albumin or a-fetoprotein promoters (Huber, et al. Proc. Natl. Acad. Sci. U.S.A. 88:8039-8043 (1991)) to target cells of liver lineage and hepatoma cells, respectively. Such tissue specific promoters can be used in retroviral vectors (Hartzoglou, et al. J. Biol. Chem. 265:17285-17293 (1990)) and adenovirus vectors (Friedman, et al. Mol. Cell. Biol. 6:3791-3797 (1986)) and still retain their tissue specificity.

Thus, there is a need for targeting expression of a interferon for the treatment of cancer, hepatitis, and other conditions amenable to therapy with a interferon. The instant invention addresses this need, and more.

### SUMMARY OF THE INVENTION

One aspect of the invention is a method for providing a patient with an interferon α polypeptide comprising introducing into a tissue of interest of the patient a vector comprising a nucleic acid segment encoding an interferon α polypeptide, the nucleic acid segment being operatively linked to a promoter having specificity for the tissue of interest, wherein the polypeptide is expressed in the tissue of interest.

Another aspect of the invention is a method for increasing interferon α levels in a tissue of interest in a patient comprising introducing into the tissue of interest a vector comprising a nucleic acid segment encoding an interferon α polypeptide, the nucleic acid segment being operatively linked to a promoter having specificity for the tissue of interest, wherein the interferon a polypeptide is expressed in the tissue of interest in the patient.

Another aspect of the invention is a method for treatment of cancer responsive to interferon a comprising administering to a cancerous tissue a vector comprising a nucleic acid segment encoding an interferon α polypeptide, the nucleic acid segment encoding an interferon α polypeptide, the nucleic acid segment being operatively linked to a promoter having specificity for the tissue, wherein the α interferon polypeptide is expressed in the tissue.

Another aspect of the invention is a method of treatment of hepatitis comprising administering to a patient's liver a vector comprising a nucleic acid segment encoding an interferon α polypeptide, the nucleic acid segment being operatively linked to a promoter having specificity for liver cells, wherein the interferon α polypeptide is expressed in the patient's liver.

A further aspect of the invention is a composition comprising a vector comprising a nucleic acid segment encoding an interferon a polypeptide, the nucleic acid segment being operatively linked to a promoter having specificity for a tissue of interest.

### BRIEF DESCRIPTION OF THE DRAWINGS

FIG. 1 is a graph depicting the anti-proliferative effects of interferon a on human prostate cancer cells.

FIG. 2 is a graph depicting luciferase activity as a measure of luciferase expression driven by promoters of liver-specific genes.

FIG. 3 is an autoradiogram of the results of an experiment to determine the level and location of IFN proteins expressed in HepG2 cells following infection with recombinant adenoviruses expressing secreted and non-secreted forms of interferon. Lane 1 is the cell extract and Lane 2 is the supernatant obtained from HepG2 cells following infection with a recombinant adenovirus expressing the secreted form of interferon α2b (rAdIFNα2b). Lane 3 is the cell extract and Lane 4 is the supernatant obtained from HepG2 cells following infection with a recombinant adenovirus expressing the non-secreted form of interferon α2b (rAdNSIα2b). Lane 5is interferon α2b protein, run as a standard. A polyclonal antisera to human interferon-α was used and detection was by chemiluminescence.

FIG. 4 is a graphical illustration of the results of an experiment to determine the inhibition of cell proliferation in response to treatment with recombinant adenoviral vectors expressing non-secreted forms of interferon α2b (rAdNSIα2b) and interferon α2α1 (rAdNSIαα2α1). HepG2 (Panel A) and Hep3B (Panel B) cells were infected with increasing particle number, as indicated, with (a) a control adenovirus without an interferon transgene (rAdx, open squares), (b) rAdNSIα2b (open circles) and (c) rAdNSIα2α1 (filled circles).

FIG. 5 is a histogram illustrating that the expression of non-secretory interferons confers resistance to viral infection. Hep3B cells, uninfected (column 1), were infected with rAd-β-gal (column 2), rAdIFNα2b (column 3), rAdNSIα2b (column 4), or rAdNSIα2α1 (column 5).

FIG. 6 is a histogram depicting induction of MHC class I in response to infection of recombinant adenoviral vectors expressing secreted interferon α2b (rAdIFNα2b) and non-secreted interferon α2b (rAdNSIα2b). Hep 3B cells, uninfected (column 1), or those infected with a control virus (rAdx, column 2), rAdIFNα2b (column 3) or rAdNSIα2b (column 4) were stained with a PE conjugated antibody for human MHC class I. The mean fluorescence was measured and is plotted on the vertical axis.

FIG. 7 is an autoradiogram to measure the phosphorylation of STAT1 in response to infection of recombinant adenoviral vectors expressing secreted and non-secreted interferon s. HepG2 cells, uninfected (lane 1), or following 12 hr of infection with rAdIFNα2b (lane 2), rAdNSIα2b (lane 3), rAdIFNα2α1 (lane 4), rAdNSIα2α1 (lane 5), rAd-βgal (lane 6) were used to immunoprecipitate with STAT1 antibody. The immunoprecipitates were electrophoresed and probed with a phosphotyrosine antibody. The data presented demonstrates that the non-secreted and secreted forms of interferon are capable of inducing the phosphorylation of STAT1.

FIG. 8 is an autoradiogram to measure the levels of the cell cycle regulatory proteins, Rb and p21 in response to infection of recombinant adenoviral vectors expressing secreted and non-secreted interferon in HepG2 cells. Uninfected HepG2 cells (lane 1), or those infected for 12 hr with rAd-βgal (lane 2), rAdIFNα2b (lane 3), rAdNSIα2b (lane 4), rAdIFNα2α1 (lane 5), rAdNSIα2α1 (lane 6) were used. The cell extracts were electrophoresed and probed with antibodies specific for retinoblastoma protein (Panel A) or p21 (Panel B).

FIG. 9 is a graphical representation of data generated to demonstrate the specificity of AFP promoter as a function of AFP status of the cell. AFP positive HepG2 cells (Panel A) and AFP negative HLF cells (Panel B) were exposed to recombinant adenoviral vectors expressing secreted forms of interferon α2b (rAdIFNα2b) and α2α1 (rAdIFNα1α2). After 48 hr of infection, supernatants from these cells were added to PC-3 cells and allowed to grow for 48 hr. The survival of PC-3 cells was measured using the MTT reagent (Boehringer-Mannheim, Indianpolis, Ind.) and by measuring the absorption at 560 nm. As can be seen from the data presented, the expression of IFN was preferential to those cells which are positive for AFP. Column 1 in both panels represents uninfected control cells. Column 2 in both panels represents infection with a control adenovirus with no interferon transgene (rAdx ). Column 3 in both panels represents infection with a recombinant adenovirus expressing secreted interferon α2b under control of the AFP promoter. Column 4 in both panels represents infection with a recombinant adenovirus expressing secreted interferon α2α1 under control of the AFP promoter.

FIG. 10 is a graphical presentation of the results of an in vivo experiment to determine the anti-tumor effects of recombinant adenoviruses expressing secreted and non-secreted forms of interferon α2b in a nude mouse xenograft tumor model. The vertical axis is a representation of tumor size in cubic millimeters. The horizontal axis is a measure of time in days following injection of the tumor cells into the animal. The arrows (a) indicate the times of intratumoral injection of 1×10¹⁰ particles of the recombinant virus. As can be seen from the data presented, recombinant adenoviral vectors expressing non-secreted and secreted forms of interferon α2b possess anti-tumor activity in vivo.

### DETAILED DESCRIPTION OF THE INVENTION

The instant invention provides methods for the tissue specific expression of IFN-α using tissue specific promoters. The term IFN-α as used herein is intended to include all subclasses of interferon cc, deletion, insertion, or substitution variants thereof, biologically active fragments, and allelic forms. “Biologically active” as used herein refers to any anti-viral or anti-proliferative activity as measured by techniques well known in the art (see, for example, Openakker, et al., supra; Mossman J. Immunol. Methods 65:55 (1983)). Recombinant interferon as have been cloned and expressed in E. coli by several groups (for example, Weissmann, et al. Science 209:1343-1349 (1980); Sreuli, et al. Science 209:1343-1347 (1980); Goeddel, et al. Nature 290:20-26 (1981); Henco, et al. J. Mol. Biol. 185:227-260 (1985)). Preferably, the interferon cc is interferon α2a or 2b (see, for example, WO 91/18927), although any interferon cc may be used.

Nucleic acids encoding the IFN-α polypeptide can be DNA or RNA. The phrase “nucleic acid sequence encoding” refers to a nucleic acid which directs the expression of a specific protein or peptide. The nucleic acid sequences include both the DNA strand sequence that is transcribed into RNA and the RNA sequence that is translated into protein. The nucleic acid sequences include both the fall length nucleic acid sequences as well as non-full length sequences derived from the full length protein. It is further understood that the sequence includes the degenerate codons of the native sequence or sequences which may be introduced to provide codon preference in a specific host cell.

The term “vector”, refers to viral expression systems, autonomous self-replicating circular DNA (plasmids), and includes both expression and non-expression plasmids. Where a recombinant microorganism or cell culture is described as hosting an “expression vector,” this includes both extrachromosomal circular DNA and DNA that has been incorporated into the host chromosome(s). Where a vector is being maintained by a host cell, the vector may either be stably replicated by the cells during mitosis as an autonomous structure, or is incorporated within the host's genome. A vector contains multiple genetic elements positionally and sequentially oriented, i.e., operatively linked with other necessary elements such that nucleic acid in the vector encoding IFN-α can be transcribed, and when necessary, translated in transfected cells.

The term “gene” as used herein is intended to refer to a nucleic acid sequence which encodes a polypeptide. This definition includes various sequence polymorphisms, mutations, and/or sequence variants wherein such alterations do not affect the function of the gene product. The term “gene” is intended to include not only coding sequences but also regulatory regions such as promoters, enhancers, and termination regions. The term further includes all introns and other DNA sequences spliced from the MRNA transcript, along with variants resulting from alternative splice sites.

The term “plasmid” refers to an autonomous circular DNA molecule capable of replication in a cell, and includes both the expression and non-expression types. Where a recombinant microorganism or cell culture is described as hosting an “expression plasmid”, this includes both extrachromosomal circular DNA molecules and DNA that has been incorporated into the host chromosome(s). Where a plasmid is being maintained by a host cell, the plasmid is either being stably replicated by the cells during mitosis as an autonomous structure or is incorporated within the host's genome.

The phrase “recombinant protein” or “recombinantly produced protein” refers to a peptide or protein produced using non-native cells that do not have an endogenous copy of DNA able to express the protein. The cells produce the protein because they have been genetically altered by the introduction of the appropriate nucleic acid sequence. The recombinant protein will not be found in association with proteins and other sub-cellular components normally associated with the cells producing the protein. The terms “protein” and “polypeptide” are used interchangeably herein.

In general, the IFN-α is provided in an expression vector comprising the following elements linked sequentially at appropriate distances for functional expression: a tissue-specific promoter, an initiation site for transcription, a 3′ untranslated region, a 5′ mRNA leader sequence, a nucleic acid sequence encoding an a interferon polypeptide, and a polyadenylation signal. Enhancer sequences and other sequences aiding expression and/or secretion can also be included in the expression vector. Additional genes, such as those encoding drug resistance, can be included to allow selection or screening for the presence of the recombinant vector. Such additional genes can include, for example, genes encoding neomycin resistance, multi-drug resistance, thymidine kinase, β-galactosidase, dihydrofolate reductase (DHFR), and chloramphenicol acetyl transferase.

In one preferred embodiment of the present invention, the interferon is expressed intracellularly by the use of an expression vector containing an interferon-α polypeptide lacking a secretion leader sequence. As previously indicated, recombinant interferon-α2b protein is regularly administered for the treatment of hepatocellular carcinoma in human beings. However, the treatment of hepatocellular carcinoma with interferon-α2b protein requires large doses of protein which can produce undesirable side effects in some human subjects. Normally, interferons are cytokines which are expressed and secreted from the producer cell and their effects mediated through a cell surface receptor mechanism. However, as can be seen from the data presented below, interferon-α produced within a cell absent a secretion signal peptide sequence is capable of mediating the full effects of interferon-α and bypassing the cell surface receptor mechanism. Because gene therapy vectors are capable of producing large quantities of transgenes over a long period of time, in some subjects it may be desirable to minimize any associated toxicity with expressed transgene. The present invention addresses this need by providing recombinant adenoviral vectors which express interferon proteins capable of exerting a therapeutic effect within the target cell. This is particularly advantageous in the treatment and elimination of cancerous cells because once the intracellularly expressed interferon protein kills the cancer cell, there is only a minimal release of interferon protein to the surrounding tissue (which is advantageous) and once all infected cells are eliminated, the potential side effects of long term systemic expression of high levels of interferon are similarly eliminated.

The following provides a description of experiments which demonstrate that interferons expressed intracellularly from recombinant gene therapy vectors demonstrate the full activity of normal (i.e. secreted) interferon species. A series of recombinant adenoviral vectors expressing secreted and non-secreted forms of interferon-ac and appropriate control vectors were prepared in substantial accordance with the teaching of Example 4 herein. For purposes of discussion, some of the properties of these vectors are summarized in Table 1 below:

TABLE 1
Recombinant Adenoviral Vectors

| | | | Secretion |
|---|---|---|---|
| Name | Promoter | Transgene | Leader |
| rAdx | AFP | none | n/a |
| rAdNSIα2b | AFP | interferon-α2b | no |
| rAdIFNα2b | AFP | interferon-α2b | yes |
| rAdNSIα2α1 | AFP | interferon-α2α1 | no |
| rAdIFNα2α1 | AFP | interferon-α2α1 | yes |
| rAdβgal | CMV | β-galactosidase | no |

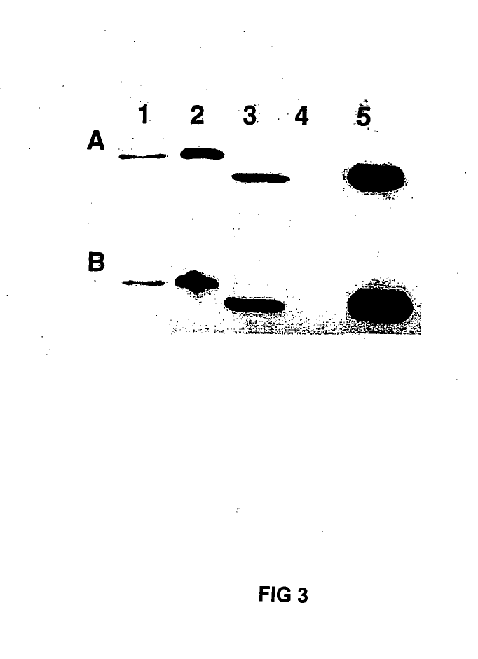

First, experiments were conducted to insure that the interferon expressed in the absence of a secretion leader sequence was located intracellularly and was not secreted by some other mechanism. Briefly, HepG2 cells were infected with the above viruses and the cell extracts and supernatants were analyzed by Western blotting. The results are presented in FIG. 3 of the attached drawings. As can be seen from the data presented, the synthesis and secretion of interferon is observed in response to infection with rAdIFNα2b and rAdIFNα2α1. In contrast, infection with rAdNSIα2b and rAdNSIα2α1, interferon is seen in the cell extracts and none is observed in the supernatants. This was further verified by immunoassay. Cell extracts and supernatants from HepG2 cells infected for 48 hr with recombinant adenoviruses expressing secreted and non-secreted interferons. An immunoassay procedure performed in accordance with the teaching of Example 5 herein was used to measure the amount of interferon protein produced. The results are presented in Table 2 below:

TABLE 2
Quantity and Localization of IFNα2b in Response to
Infection with recombinant adenoviruses containing IFNα2b
with and without a secreted leader sequence

| | Cell Extract | |
|---|---|---|
| rAd - Construct | (ng/ml) | Supernatant |
| rAd-IFNα2b | 13.5 | 35.6 |
| rAd-NAIα2b | 19.4 | <LOQ |
| rAd-β-gal | <LOQ | <LOQ |
| Uninfected Control | <LOQ | <LOQ |

* <LOQ indicates that the levels were below the limit of quantitation of 0.1 ng/ml.

From the data presented above, it is clear that the interferon is produced and secreted in rAdIFNα2b infected cells. However, cells infected with the non-secreted, rAdNSIα2b construct, the interferon produced was retained within the cell and the supernatant had no detectable interferon, as was the case with uninfected on rAd-β-gal infected virus. Consequently, the interferon expressed from the recombinant adenoviral vectors in the absence of secretion leader sequence is located intracellularly.

Based on these results, a series of experiments was conducted in order to demonstrate that the intracellularly expressed non-secreted interferon-α possesses the characteristic activity of interferon protein administered extracellularly. The non-secreted interferons were assayed for their ability to inhibit tumor cell proliferation, antiviral effect, the ability to induce MHC-1 induction, the phosphorylation of STAT1, the accumulation of hypo-phosphorylated Rb and induction of p21.

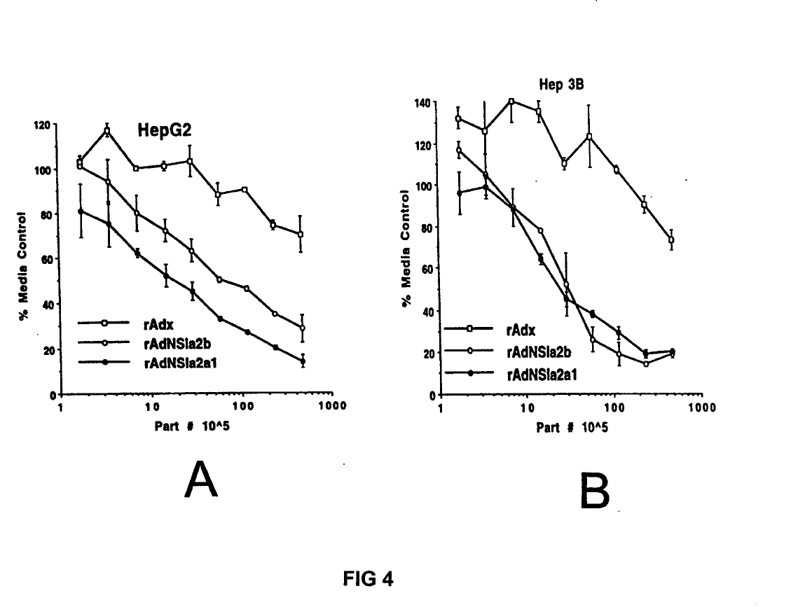

The ability of non-secreted interferon α2b or α2α1 to affect cell proliferation was tested by infecting Hep3B or HepG2 cells, which are both positive for AFP promoter. Briefly, Hep3B and HepG2 cells were infected with rAdNSIα2b and rAdNSIα2α1 and rAdx as a control. Cells were infected with increasing particle number of the control virus or the interferon expressing rAdNSIα2b or rAdNSIα2α1. After 45 hr of infection, cells were labelled with 0.5 μCi of ³H-thymidine for 3 hours and ³H-thymidine incorporation as a measure of cytopathic effect (CPE) is presented as a percentage of media control. The results are presented in FIG. 4 of the attached drawings. As can be seen from the data presented, a strong dose-dependent inhibition of cell proliferation was observed with both the non-secreted interferons α2b and α2α1.

The antiviral activity of non-secreted interferons was determined in Hep G2 cells in response to infection with rAdIFNα2b or rAdNSIα2b. The cell extracts and supernatants from HepG2 cells infected with rAdIFNα2b or rAdNSIα2b were tested for antiviral activity by using EMCV. Briefly, HepG2 cells were infected with rAdIFNα2b or rAdNSIα2b. After 48 hr of infection, cell extracts and supernatants were analyzed for anti-viral activity using EMCV. The amount of interferon was quantitated by immunoassay and the specific activity was represented as I.U./mg protein. The amount of interferon was quantitated by immunoassay and the specific activity is presented as I.U./mg protein. The results of which are presented in Table 3 below.

TABLE 3
Anti-viral activity of rAd constructs..

| | Antiviral | | Specific |
|---|---|---|---|
| | Activity | Concentration | Activity |
| Construct/Test Sample | (IU/ml) | (ng/ml) | (IU/mg) |
| rAd-IFNα2b/cell extract | 6882 | 13.5 | 5.08 × 10⁸ |
| rAd-IFNα2b/supernatant | 19680 | 41.7 | 4.76 × 10⁸ |
| rAd-NSIα2b/cell extract | 9344 | 19.4 | 4.82 × 10⁸ |
| rAd-NSIα2b/supernatant | <LOQ | <LOQ | <LOQ |

As can be seen from the data presented, with the virus expressing the secreted interferon (rAdIFNα2b), antiviral activity was observed in both the cell extract and the supernatant. However, the virus expressing the non-secereted interferon (rAdNSIα2b), the antiviral activity was restricted to the cell extract, while the supernatant had no detectable antiviral activity.

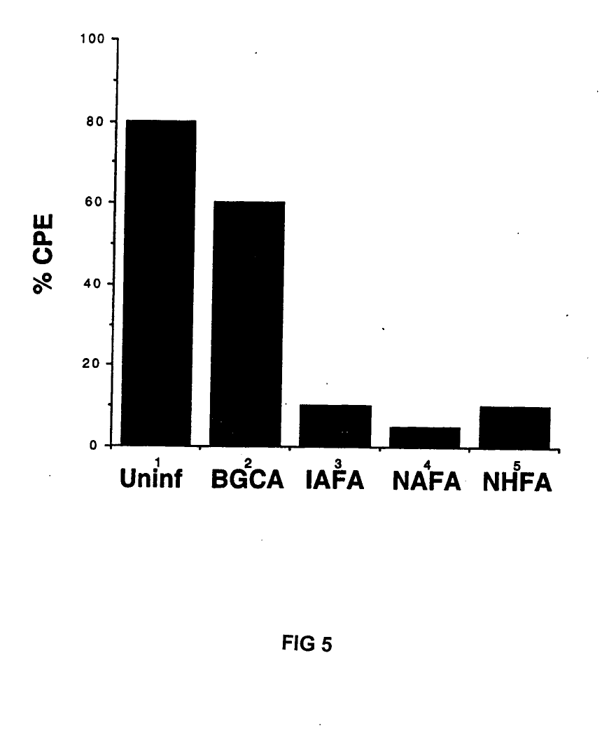

To demonstrate that the expression of non-secreted interferon confers antiviral properties on intact cells, Hep 3B cells were exposed to virus for 1 hour and rinsed. Cells were allowed to grow for an additional 12 hours. At that time, the cell populations were exposed to EMCV for 1 hour, excess virus removed by washing with media, and the media replaced. The number of plaques was counted after two days following EMCV exposure. The results are presented in FIG. 5 of the attached drawings and antiviral effect expressed as a percentage of CPE of media control. As can be seen from the data presented, the expression of non-secreted interferons α2b or α2α1 makes these cells resistant to infection by EMCV comparable to the secreted interferon.

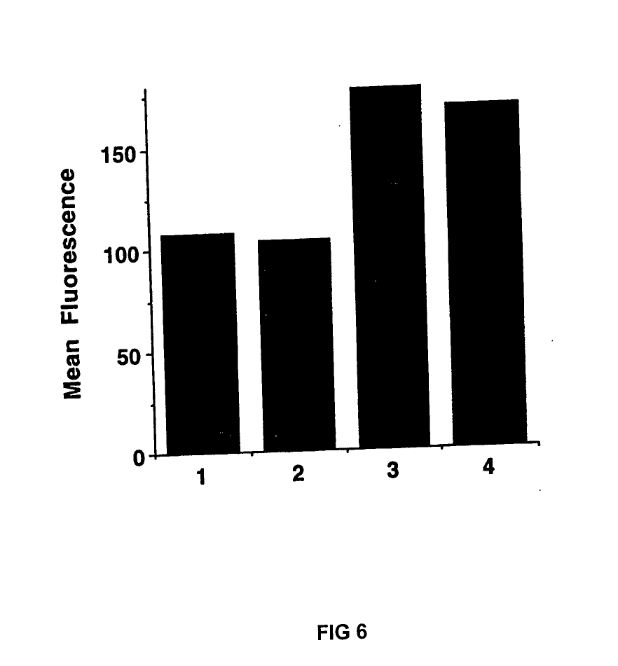

Induction of major histocompatiability complex I is one of the important properties of interferon. The ability of the The induction of MHC I seen with the secreted interferon is also seen with the non-secreted adenovirus construct, while there was no increase with the control virus (FIG. 6). The data shows that secreted and non-secreted interferons expressed from a recombinant adenoviral vector possess a similar ability to induce MHC class I.

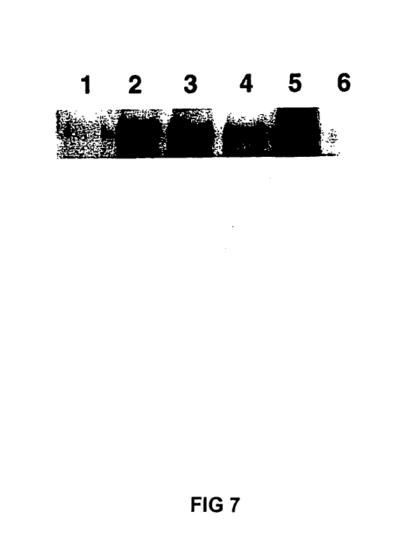

Having ascertained that the non-secreted interferons exhibited the biological activity characteristic of their secreted counterparts, experiments were performed to test if the signalling events in these two situations are comparable. Phosphorylation of STAT1 is one of the early events in the signaling of interferons. Cell extracts from HepG2 cells, either uninfected or those infected for 12 hr with rAdβgal, rAdIFNα2b, rAdOFMα2α1, rAdNSIα2b, and rAdNSIα2α1 were immunoprecipitated with a STAT1 antibody, electrophoresed and probed with a phosphotyrosine specific antibody. The results are presented in FIG. 7 of the attached drawings. As can be seen from the data presented, there was no detectable phosphorylation of STAT1 in either uninfected or rAdβgal, whereas for both the interferons α2b and α2α1 in secreted or non-secreted form, a strong phosphorylation of STAT1 observed.

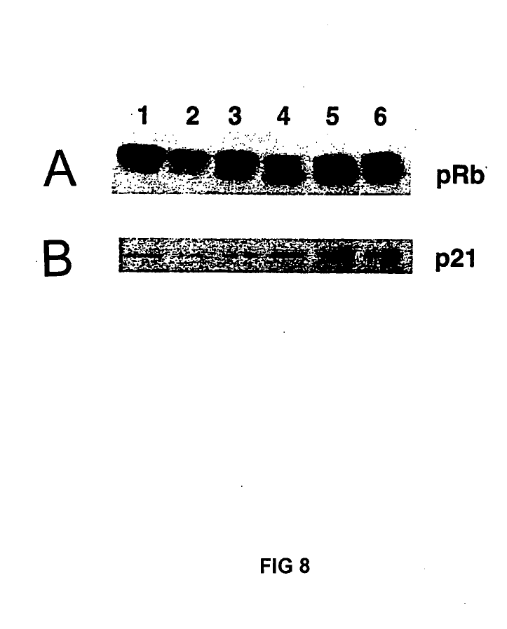

The presence of hypophosphorylated Rb is associated with the inhibition of cell proliferation. The growth inhibitory properties of interferon have been suggested to be mediated through the modification of phosphorylation status of retinoblastoma protein (pRb). In order to determine the effects on Rb phosphorylation in response to secreted and non-secreted interferon, the cell extracts from uninfected HepG2 cells or those infected with a control or interferon expressing virus were electrophoresed and probed with a monoclonal antibody for pRb. The results are presented in FIG. 8 of the attached drawings. As can be seen from the data presented, the uninfected and b-gal expressing virus, phosphorylated pRb was the most predominant band observed. For the interferons α2b and α2αα1, in the secreted or non-secreted state, a greater accumulation of the hypophosphorylated pRb was observed. Consequently, the effect on Rb phosphorylation was observed in response to both species. Additionally, interferon has been shown to interact with the cell cycle regulatory protein p21. The cell extracts from HepG2 cells infected with different viruses were probed with the antibody for p21 and the data presented in FIG. 8 of the attached drawings. As can be seen from the data presented, there is greater accumulation of p21 in response to secreted and non-secreted interferons.

In the instant invention, targeting of the IFN-α to a particular tissue of interest is accomplished by the use of a promoter and/or other expression elements preferentially used by the tissue of interest. Examples of known tissue-specific promoters include the promoter for creatine kinase, which has been used to direct the expression of dystrophin cDNA expression in muscle and cardiac tissue (Cox, et al. Nature 364:725-729 (1993)); immunoglobulin heavy or light chain promoters for the expression of genes in B cells; albumin or α-fetoprotein promoters to target cells of liver lineage and hepatoma cells, respectively.

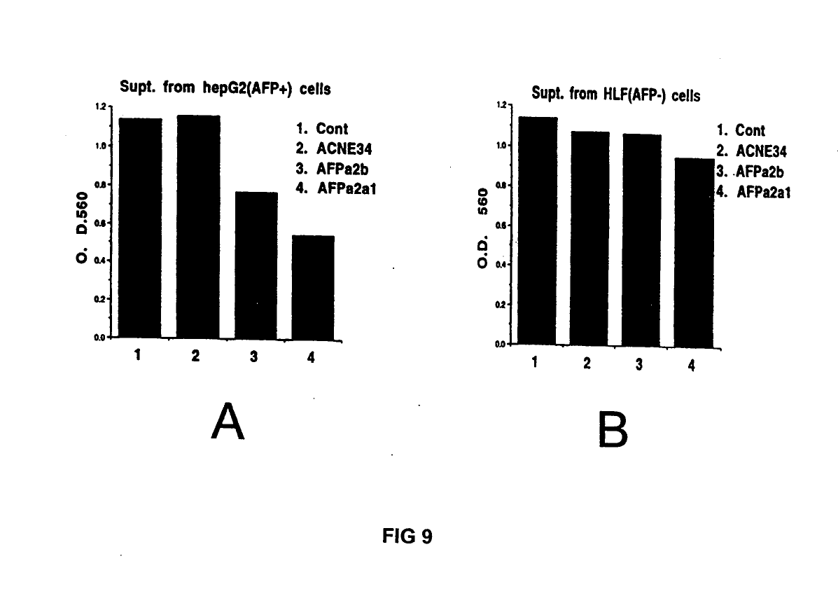

In an exemplary embodiment of the invention, recombinant adenoviral vectors encoding interferons α2b (rAdIFNα2b) or α2α1 (rAdIFNα2α1) under the control of α-feto protein promoter (AFP) promoter were constructed and characterized. The recombinant adenoviral vectors were prepared in substantial accordance with the teaching of Example 4 herein. In order to demonstrate the selective expression of the transgene from the AFP promoter, Hep G2 (AFP positive) or HLF (AFP negative) cells were infected with rAdIFNα2b or rAdOFMα2α1 for 48 hours. Interferon activity in the supernatants of these cells was assayed by the ability of the supernatants to inhibit cell proliferation characteristic of interferon. An aliquot of the supernatants from the infected HepG2 or HLF cells was added to PC3 (human prostate cancer) cells. The growth of PC3 cells was followed by using MTT reagent (commercially available from Boehringer-Mannheim). The results ate presented in FIG. 9 of the attached drawings. As can be seen from the data presented, the effect of the cell supernatant on the growth of PC3 cells from HepG2 cells infected with rAdIFNα2b and rAdOFMα2α1 demonstrates that these supernatants were capable of inhibiting the growth of PC3 cells. In contrast, supernatants obtained from HLF cells infected with rAdIFNα2b and rAdIFNα2α1 had essentially no effect on the growth of PC3 cells. Lanes 1 and 2 in each panel of FIG. 9 are controls and represent the effect on PC3 cells of supernatants from HepG2 and HLF cells uninfected with virus and infected with a control virus containing the vector backbone without a transgene, respectively.

In addition to the ability to prevent cell proliferation, interferon-α is capable of conferring resistance to viral infection. The activity of interferon in the supernatants from HepG2 and HLF cells from the above experiment were also assayed for their ability to inhibit EMCV infection in A549 (human lung cancer) cells. The results are presented in FIG. 5 of the attached drawings. As can be seen from the data presented, supernatants of HepG2 cells infected with rAdIFNα2b and rAdIFNα2α1 in produced 4.8×10³ and 2.4×10³ I.U./ml of interferon-α2b and α2α1, respectively. However, the supernatants from HLF cells infected with rAdIFNα2b and rAdOFMα2α1 was 9 and 19 I.U./ml. These results further demonstrate the ability of the AFP promoter to restrict transgene expression essentially only in AFP expressing cells.

As can be seen from the data presented above, the expression of AFP promoter was restricted to the cells that are positive for this promoter. AFP promoter driven constructs were expressed specifically in cells that were expressing this promoter. AFP promoter is activated in 70-80% of hepatocellular carcinomas. Since AFP promoter is specifically turned on in hepatocellular carcinoma, these recombinant adenoviral constructs which express non-secreted interferon under control of the AFP promoter are particularly useful in delivering interferon genes to the liver tumor cells while reducing toxicity to the neighboring tissues.

Exemplary tissue-specific expression elements for the liver include but are not limited to HMG-CoA reductase promoter (Luskey, Mol. Cell. Biol. 7(5):1881-1893 (1987)); sterol regulatory element 1 (SRE-1; Smith, et al. J. Biol. Chem. 265(4):2306-2310 (1990); phosphoenol pyruvate carboxy kinase (PEPCK) promoter (Eisenberger, et al. Mol. Cell Biol. 12(3):1396-1403 (1992)); human C-reactive protein (CRP) promoter (Li, et al. J. Biol. Chem. 265(7):4136-4142 (1990)); human glucokinase promoter (Tanizawa, et al. Mol. Endocrinology 6(7):1070-81 (1992); cholesterol 7-α hydroylase (CYP-7) promoter (Lee, et al. J. Biol. Chem. 269(20):14681-9 (1994)); β-galactosidase α-2,6 sialyltransferase promoter (Svensson, et al. J. Biol. Chem. 265(34):20863-8 (1990); insulin-like growth factor binding protein (IGFBP-1) promoter (Babajko, et al. Biochem Biophys. Res. Comm. 196 (1):480-6 (1993)); aldolase B promoter (Bingle, et al. Biochem J. 294(Pt2):473-9 (1993)); human transferrin promoter (Mendelzon, et al. Nucl. Acids Res. 18(19):5717-21 (1990); collagen type I promoter (Houglum, et al. J. Clin. Invest. 94(2):808-14 (1994)).

Exemplary tissue-specific expression elements for the prostate include but are not limited to the prostatic acid phosphatase (PAP) promoter (Banas, et al. Biochim. Biophys. Acta. 1217(2):188-94 (1994); prostatic secretory protein of 94 (PSP 94) promoter (Nolet, et al. Biochim. Biophys. ACTA 1098(2):247-9 (1991)); prostate specific antigen complex promoter (Casper, et al. J. Steroid Biochem. Mol. Biol. 47 (1-6):127-35 (1993)); human glandular kallikrein gene promoter (hgt-1) (Lilja, et al. World J. Urology 11(4):188-91 (1993). Exemplary tissue-specific expression elements for gastric tissue include but are not limited to the human H⁺/K⁺-ATPase α subunit promoter (Tanura, et al. FEBS Letters 298:(2-3): 137-41 (1992)).

Exemplary tissue-specific expression elements for the pancreas include but are not limited to pancreatitis associated protein promoter (PAP) (Dusetti, et al. J. Biol. Chem. 268(19):14470-5 (1993)); elastase 1 transcriptional enhancer (Kruse, et al. Genes and Development 7(5):774-86 (1993)); pancreas specific amylase and elastase enhancer promoter (Wu, et al. Mol. Cell. Biol. 11(9):4423-30 (1991); Keller, et al. Genes & Dev. 4(8):1316-21 (1990)); pancreatic cholesterol esterase gene promoter (Fontaine, et al. Biochemisty 30(28):7008-14 (1991)).

Exemplary tissue-specific expression elements for the endometrium include but are not limited to the uteroglobin promoter (Helftenbein, et al. Annal. NY Acad. Sci. 622:69-79 (1991)).

Exemplary tissue-specific expression elements for adrenal cells include but are not limited to cholesterol side-chain cleavage (SCC) promoter (Rice, et al. J. Biol. Chem. 265:11713-20 (1990).

Exemplary tissue-specific expression elements for the general nervous system include but are not limited to y-y enolase (neuron-specific enolase, NSE) promoter (Forss-Petter, et al. Neuron 5(2):187-97 (1990)).

Exemplary tissue-specific expression elements for the brain include but are not limited to the neurofilament heavy chain (NF-H) promoter (Schwartz, et al. J. Biol. Chem. 269(18):13444-50 (1994)).

Exemplary tissue-specific expression elements for lymphocytes include but are not limited to the human CGL-1/granzyme B promoter (Hanson, et al. J. Biol. Chem. 266 (36):24433-8 (1991)); the terminal deoxy transferase (TdT), lambda 5, VpreB, and 1ck (lymphocyte specific tyrosine protein kinase p561ck) promoter (Lo, et al. Mol. Cell. Biol. 11(10):5229-43 (1991)); the humans CD2 promoter and its 3′transcriptional enhancer (Lake, et al. EMBO J. 9(10):3129-36 (1990)), and the human NK and T cell specific activation (NKG5) promoter (Houchins, et al. Immunogenetics 37(2):102-7 (1993)).

Exemplary tissue-specific expression elements for the colon include but are not limited to pp60c-src tyrosine kinase promoter (Talamonti, et al. J. Clin. Invest 91(1):53-60 (1993)); organ-specific neoantigens (OSNs), mw 40 kDa (p40) promoter (Ilantzis, et al. Microbiol. Immunol. 37(2):119-28 (1993)); colon specific antigen-P promoter (Sharkey, et al. Cancer 73(3 supp.) 864-77 (1994)).

Exemplary tissue-specific expression elements for breast cells include but are not limited to the human α-lactalbumin promoter (Thean, et al. British J. Cancer. 61(5):773-5 (1990)).

Other elements aiding specificity of expression in a tissue of interest can include secretion leader sequences, enhancers, nuclear localization signals, endosmolytic peptides, etc. Preferably, these elements are derived from the tissue of interest to aid specificity.

Techniques for nucleic acid manipulation of the nucleic acid sequences of the invention such as subcloning nucleic acid sequences encoding polypeptides into expression vectors, labelling probes, DNA hybridization, and the like are described generally in Sambrook, et al., Molecular Cloning-A Laboratory Manual (2nd Ed.), Vol. 1-3, Cold Spring Harbor Laboratory, Cold Spring Harbor, N.Y., (1989), which is incorporated herein by reference. This manual is hereinafter referred to as “Sambrook, et al.”

Once DNA encoding a sequence of interest is isolated and cloned, one can express the encoded proteins in a variety of recombinantly engineered cells. It is expected that those of skill in the art are knowledgeable in the numerous expression systems available for expression of DNA encoding. No attempt to describe in detail the various methods known for the expression of proteins in prokaryotes or eukaryotes is made here.

In brief summary, the expression of natural or synthetic nucleic acids encoding a sequence of interest will typically be achieved by operably linking the DNA or cDNA to a promoter (which is either constitutive or inducible), followed by incorporation into an expression vector. The vectors can be suitable for replication and integration in either prokaryotes or eukaryotes. Typical expression vectors contain transcription and translation terminators, initiation sequences, and promoters useful for regulation of the expression of polynucleotide sequence of interest. To obtain high level expression of a cloned gene, it is desirable to construct expression plasmids which contain, at the minimum, a strong promoter to direct transcription, a ribosome binding site for translational initiation, and a transcription/translation terminator. The expression vectors may also comprise generic expression cassettes containing at least one independent terminator sequence, sequences permitting replication of the plasmid in both eukaryotes and prokaryotes, i.e., shuttle vectors, and selection markers for both prokaryotic and eukaryotic systems. See Sambrook, et al.

The constructs of the invention can be introduced into the tissue of interest in vivo or ex vivo by a variety of methods. In some embodiments of the invention, the vector is introduced to cells by such methods as microinjection, calcium phosphate precipitation, liposome fusion, or biolistics. In further embodiments, the DNA is taken up directly by the tissue of interest. In other embodiments, the constructs are packaged into a viral vector system to facilitate introduction into cells.

Viral vector systems useful in the practice of the instant invention include adenovirus, herpesvirus, adeno-associated virus, minute virus of mice (MVM), HIV, sindbis virus, and retroviruses such as Rous sarcoma virus, and MoMLV. Typically, the constructs of the instant invention are inserted into such vectors to allow packaging of the interferon expression construct, typically with accompanying viral DNA, infection of a sensitive host cell, and expression of the interferon-α gene. In one embodiment of the invention as exemplified herein, the vector is a viral vector. The viral genomes may be modified by conventional recombinant DNA techniques to provide expression of interferon-α and may be engineered to be replication deficient, conditionally replicating or replication competent. Chimeric viral vectors which exploit advantageous elements of each of the parent vector properties (See e.g., Feng, et al.(1997) Nature Biotechnology 15:866-870) may also be useful in the practice of the present invention. Minimal vector systems in which the viral backbone contains only the sequences needed for packaging of the viral vector and may optionally include an interferon-α expression cassette may also be employed in the practice of the present invention. In some instances it may be advantageous to use vectors derived from different species from that to be treated which possess favorable pathogenic features such as avoidance of pre-existing immune response. For example, equine herpes virus vectors for human gene therapy are described in WO 98/27216 published Aug. 5, 1998. The vectors are described as useful for the treatment of humans as the equine virus is not pathogenic to humans. Similarly, ovine adenoviral vectors may be used in human gene therapy as they are claimed to avoid the antibodies against the human adenoviral vectors. Such vectors are described in WO 97/06826 published Apr. 10, 1997.

In one embodiment of the invention as exemplified herein, the vector is an adenoviral vector. The term adenoviral vector refers collectively to animal adenoviruses of the genus mastadenovirus including but no limited to human, bovine, ovine, equine, canine, porcine, murine and simian adenovirus subgenera. In particular, human adenoviruses includes the A-F sugenera as well as the individual serotypes thereof the individual serotypes and A-F subgenera including but not limited to human adenovirus types 1, 2, 3, 4, 4a, 5, 6, 7, 8, 9, 10, 11 (Ad11A and Ad11P), 12, 13, 14, 15, 16, 17, 18, 19, 19a, 20, 21, 22, 23, 24, 25, 26, 27, 28, 29, 30, 31, 32, 33, 34, 34a, 35, 35p, 36, 37, 38, 39, 40, 41, 42, 43, 44, 45, 46, 47, 48, and 91. The term bovine adenoviruses includes but is not limited to bovine adenovirus types 1, 2, 3, 4, 7, and 10. The term canine adenoviruses includes but is not limited to canine types 1 (strains CLL, Glaxo, R1261, Utrect, Toronto 26-61) and 2. The term equine adenoviruses includes but is not limited to equine types 1 and 2. The term porcine adenoviruses includes but is not limited to porcine types 3 and 4. The use of adenoviral vectors for the delivery of exogenous transgenes are well known in the art. See e.g., Zhang, W-W. (1999) Cancer Gene Therapy 6:113-138. A preferred embodiment of an adenoviral vector for expression of the inteferon-α sequence is a replication deficient human adenovirus of serotype 2 or 5 created by elimination of adenoviral E1 genes resulting in a virus which is substantially incapable of replicating in cells which do not complement the E1 functions. A particularly advantageous vector is the adenovirus vector disclosed by Wills, et al. Hum. Gene Therapy 5:1079-1088 (1994).

In still other embodiments of the invention, the recombinant IFN-α constructs of the invention are conjugated to a cell receptor ligand for facilitated uptake (e.g., invagination of coated pits and internalization of the endosome) through a DNA linking moiety (Wu, et al. J. Biol. Chem. 263:14621-14624 (1988); WO 92/06180). For example, the DNA constructs of the invention can be linked through a polylysine moiety to asialo-oromucocid, which is a ligand for the asialoglycoprotein receptor of hepatocytes.

Similarly, viral envelopes used for packaging the constructs of the invention can be modified by the addition of receptor ligands or antibodies specific for a receptor to permit receptor-mediated endocytosis into specific cells (e.g., WO 93/20221, WO 93/14188; WO 94/06923). Cell type specificity or cell type targeting may also be achieved in vectors derived from viruses having characteristically broad infectivities such as adenovirus by the modification of the viral envelope proteins. For example, cell targeting has been achieved with adenovirus vectors by selective modification of the viral genome knob and fiber coding sequences to achieve expression of modified knob and fiber domains having specific interaction with unique cell surface receptors. Examples of such modifications are described in Wickham, et al. (1997) J. Virol. 71(11):8221-8229 (incorporation of RGD peptides into adenoviral fiber proteins); Arnberg, et al. (1997) Virology 227:239-244 (modification of adenoviral fiber genes to achieve tropism to the eye and genital tract); Harris and Lemoine (1996) TIG 12(10):400-405; Stevenson, et al. (1997) J. Virol. 71(6):4782-4790; Michael, et al.(1995) gene therapy 2:660-668 (incorporation of gastrin releasing peptide fragment into adenovirus fiber protein); and Ohno, et al. (1997) Nature Biotechnology 15:763-767 (incorporation of Protein A-IgG binding domain into Sindbis virus). Other methods of cell specific targeting have been achieved by the conjugation of antibodies or antibody fragments to the envelope proteins (see, e.g. Michael, et al. (1993) J. Biol. Chem. 268:6866-6869, Watkins, et al. (1997) Gene Therapy 4:1004-1012; Douglas, et al. (1996) Nature Biotechnology 14: 1574-1578. Viral vectors encompassing one or more of such targeting modifications may optionally be employed in the practice of the present invention to enhance the selective infection and expression of In some embodiments of the invention, the DNA constructs of the invention are linked to viral proteins, such as adenovirus particles, to facilitate endocytosis (Curiel, et al. Proc. Natl. Acad. Sci. U.S.A. 88:8850-8854 (1991)). In other embodiments, molecular conjugates ofthe instant invention can include microtubule inhibitors (WO/9406922); synthetic peptides mimicking influenza virus hemagglutinin (Plank, et al. J. Biol. Chem. 269:12918-12924 (1994)); and nuclear localization signals such as SV40 T antigen (WO93/19768).

The term “treatment” as used herein is intended to refer to the introduction of nucleic acid encoding an cc interferon to a patient for the purpose of exposing a tissue of interest, especially a tissue having one or more cells demonstrating some pathology, to cc interferon. Thus, for example, a “cancerous” tissue is intended to refer to a tissue in which one or more cells is classified as cancerous, malignant, tumorous, precancerous, transformed, or as an adenoma or carcinoma, or any other synonym commonly used in the art for these conditions.

A “noncancerous” cell as used herein is understood in the art as excluded from the definition of cancerous or cancer cell, and can include normal cells and cells displaying some pathological feature such as infection by a virus, bacterium, parasite, or other organism, cells affected by a hereditable condition that renders them less optimal than normal or wild type counterparts, cells affected by some presumed non-infectious disease state such as diabetes, etc., and cells which have survived any of these stresses, etc.

Treatment or therapy of any condition which would benefit from administration of IFN-α can begin prior to the diagnosis of the condition or at any time after diagnosis of a condition. Thus, for example, a patient suspected of having a precancerous lesion or an increased probability of developing some type of cancer can be treated with the compositions of the invention. Similarly, a person exposed to a pathogen, such as hepatitis B virus, can be treated with the compositions of the invention before hepatitis is diagnosed. Furthermore, suspected carriers of HBV or patients likely to become carriers can be treated after gross symptoms of the disease have improved.

The constructs of the invention are useful in the therapy of various cancers, hepatitis and other conditions in which the administration of IFN-α to raise IFN-α levels in tissues is advantageous, including but not limited to ulcerative colitis, rhinovirus infections, condyloma acuminata, laryngeal papillomitis; HIV infection, fibrosis, allergic diseases due to excess IL-4 and IgE production, and granulomatous disorders, such as Crohn's disease. Although any tissue can be targeted for which some tissue-specific expression element, such as a promoter, can be identified, of particular interest is the tissue specific administration of IFN-α to raise IFN-α levels in cancerous tissues, such as human prostate carcinoma and hepatoma tissues. Furthermore, the constructs of the invention can be used to raise IFN-α levels in tissues in pathological conditions in which non-cancerous cells are deficient in interferon production, i.e, produce less interferon than the healthy cells, such as in chronic hepatitis B virus carriers (Nouri-Aria, et al. Hepatology 14(6):1308-1311 (1991)). In some embodiments of the invention the recombinant constructs are targeted to neighboring tissues or cells to raise the local concentration of interferon a in a cell population of interest.

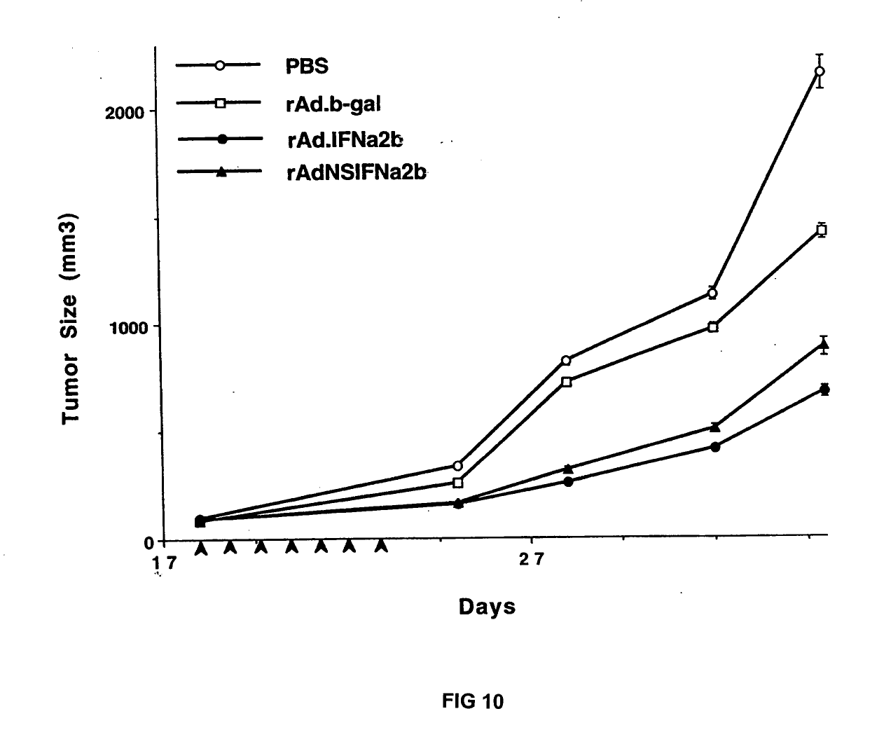

In order to demonstrate that the vectors of the present invention are useful in vivo, the recombinant adenoviral vectors in which the human interferon-α2b gene was under control of the α-feto-protein (AFP) promoter prepared in accordance with the teaching of Example 4 herein were tested for activity in vivo in a mouse xenograft tumor model. Briefly, approximately 1×10⁷ Hep3B (human hepatocellular carcinoma) cells were injected subcutaneously to generate tumors in athymic BALB/c nude mice. After the tumors were established (approximately 18 days following injection of the tumor cells), the mice were treated with daily injections of 1×10¹⁰ particles of recombinant adenoviral vectors expressing secreted and non-secreted forms of interferon α2b for seven days. A recombinant virus encoding the β-galactosidase marker gene was included to demonstrate gene specific effect. The size of the tumors was evaluated throughout the time course of the experiment and the data is presented in FIG. 10 of the attached drawings. As can be seen from the data presented, animals which received the recombinant adenoviral constructs expressing the interferon-α2b showed a significant anti-tumor effect as compared to the control vector and PBS control. The data indicates that approximately the same anti-tumor effect is demonstrated in response to secreted and non-secreted forms of interferon-α, further demonstrating that intracellular expressed interferon-α possesses the anti-tumor properties of the interferon-α protein. Therefore, AFP driven interferon adenovirus constructs, particularly the non-secreted species for the aforementioned reasons, are particularly useful in treating hepatocellular carcinoma. In addition, the blood delivery to the HCC tissue is derived mainly through the intrahepatic artery. Therefore, delivery these vectors via the intrahepatic artery (IHA administration) will provide an additional level of specificity to this approach.

The determination of the optimal dosage regimen for treatment of the disease will be based on a variety of factors which are within the discretion of the attending health care provider, such as the progression of the disease at the time of treatment, age, weight, sex, the type of vector being employed, whether it is being formulated with a delivery enhancing agent, the frequency of administration, etc. However, recombinant adenoviral vectors have been demonstrated to be safe and effective in human beings in the dosage range between 1×10⁵ and 1×10² viral particles per dose in a multiple dosing regimen over a period of several weeks. Consequently administration of recombinant adenoviral vectors encoding interferon-α may be used in such dosage ranges. In the preferred practice of the invention for the treatment of hepatocellular carcinoma in a human being, a dosage regimen comprising approximately 1×10¹⁰-1×10¹² particles of a replication deficient recombinant adenoviral vector expressing an intracellular interferon-α species is administered intratumorally or via the hepatic artery for a period of five to seven consecutive days. This dosage regimen may be repeated over a course of therapy of approximately three to six weeks. A particularly preferred dosage regimen for the treatment of hepatocellular carcinoma in a human subject suffering therefrom would be to provide intrahepatic arterial administration of from approximately 1×10¹⁰-1×10¹² particles of a replication deficient recombinant adenoviral vector expressing inteferon-α2b under control of the AFP promoter for approximately five consecutive days. Most preferably, this dosage regimen is carried out in parallel with other chemotherapeutic regimens.

In the situation where the vector is a replication competent vector, the dosage regimen may be reduced. For example, a replication competent adenoviral vector may be constructed wherein the replication is substantially restricted to hepatocellular carcinoma cells by using the AFP promoter (for example) to drive expression of E1 proteins in lieu of the native E1 promoter. Such a vector would also comprise an expression cassette comprising the interferon-α coding sequence (again, preferably lacking the secretion leader sequence) under control of the AFP promoter. Such vector would preferentially replicate in and express interferon in hepatocellular carcinoma cells and possess the desirable ability to spread to surrounding cells expanding the therapeutic effect and allowing for a reduced dosage or shorter duration of treatment.

The vectors of the present invention and pharmaceutical formulations thereof may be employed in combination with conventional chemotherapeutic agents or treatment regimens. Examples of such chemotherapeutic agents include inhibitors of purine synthesis (e.g., pentostatin, 6-mercaptopurine, 6-thioguanine, methotrexate) or pyrimidine synthesis (e.g. Pala, azarbine), the conversion of ribonucleotides to deoxyribonucleotides (e.g. hydroxyurea), inhibitors of dTMP synthesis (5-fluorouracil), DNA damaging agents (e.g. radiation, bleomycines, etoposide, teniposide, dactinomycine, daunorubicin, doxorubicin, mitoxantrone, alkylating agents, mitomycin, cisplatin, procarbazine) as well as inhibitors of microtubule function (e.g vinca alkaloids, taxol, taxotere and colchicine). Chemotherapeutic treatment regimens refers primarily to non-chemical procedures designed to ablate neoplastic cells such as radiation therapy.

The immunological response is significant to repeated in vivo administration of viral vectors. Consequently, the vectors of the present invention may be administered in combination with immunosuppressive agents. Examples of immunosuppressive agents include cyclosporine, azathioprine, methotrexate, cyclophosphamide, lymphocyte immune globulin, antibodies against the CD3 complex, adrenocorticosteroids, sulfasalzaine, FK-506, methoxsalen, and thalidomide.

The compositions of the invention will be formulated for administration by manners known in the art acceptable for administration to a mammalian subject, preferably a human. In some embodiments of the invention, the compositions of the invention can be administered directly into a tissue by injection or into a blood vessel supplying the tissue of interest. In further embodiments of the invention the compositions of the invention are administered “locoregionally”, i.e., intravesically, intralesionally, and/or topically. In other embodiments of the invention, the compositions of the invention are administered systemically by injection, inhalation, suppository, transdermal delivery, etc. In further embodiments of the invention, the compositions are administered through catheters or other devices to allow access to a remote tissue of interest, such as an internal organ. The compositions of the invention can also be administered in depot type devices, implants, or encapsulated formulations to allow slow or sustained release of the compositions.

The invention provides compositions for administration which comprise a solution of the compositions of the invention dissolved or suspended in an acceptable carrier, preferably an aqueous carrier. A variety of aqueous carriers may be used, e.g., water, buffered water, 0.8% saline, 0.3% glycine, hyaluronic acid and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

The concentration of the compositions of the invention in the pharmaceutical formulations can vary widely, i.e., from less than about 0.1%, usually at or at least about 2% to as much as 20% to 50% or more by weight, and will be selected primarily by fluid volumes, viscosities, etc., in accordance with the particular mode of administration selected.

In a preferred embodiment of the invention, particularly when the vector is a viral vector, the vector is delivered in combination or complexed with a delivery enhancing agent to increase uptake of the viral particles across the lung epithelial surface. The terms “delivery enhancers” or “delivery enhancing agents” are used interchangeably herein and includes agents which facilitate the transfer of the nucleic acid or protein molecule to the target cell. Examples of such delivery enhancing agents detergents, alcohols, glycols, surfactants, bile salts, heparin antagonists, cyclooxygenase inhibitors, hypertonic salt solutions, and acetates. Alcohols include for example the aliphatic alcohols such as ethanol, N-propanol, isopropanol, butyl alcohol, acetyl alcohol. Glycols include glycerine, propyleneglycol, polyethyleneglycol and other low molecular weight glycols such as glycerol and thioglycerol. Acetates such as acetic acid, gluconic acid, and sodium acetate are further examples of delivery-enhancing agents. Hypertonic salt solutions like 1M NaCl are also examples of delivery-enhancing agents. Bile salts such as taurocholate, sodium tauro-deoxycholate, deoxycholate, chenodesoxycholate, glycocholic acid, glycochenodeoxycholic acid and other astringents such as silver nitrate may be used. Heparin-antagonists like quaternary amines such as protamine sulfate may also be used. Anionic, cationic, zwitterionic, and nonionic detergents may also be employed to enhance gene transfer. Exemplary detergents include but are not limited to taurocholate, deoxycholate, taurodeoxycholate, cetylpyridium, benalkonium chloride, Zwittergent 3-14 detergent, CHAPS (3-[(3-Cholamidopropyl) dimethylammoniol]-1-propanesulfonate hydrate), Big CHAP, Deoxy Big CHAP, Triton-X-100 detergent, C12E8, Octyl-B-D-Glucopyranoside, PLURONIC- F68 detergent, Tween 20 detergent, and TWEEN 80 detergent (CalBiochem Biochemicals).

The compositions of the invention may also be administered via liposomes. Liposomes include emulsions, foams, micelles, insoluble monolayers, liquid crystals, phospholipid dispersions, lamellar layers and the like. In these preparations the composition of the invention to be delivered is incorporated as part of a liposome, alone or in conjunction with a molecule which binds to a desired target, such as antibody, or with other therapeutic or immunogenic compositions. Thus, liposomes either filled or decorated with a desired composition of the invention of the invention can delivered systemically, or can be directed to a tissue of interest, where the liposomes then deliver the selected therapeutic/immunogenic peptide compositions.

Liposomes for use in the invention are formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka, et al. Ann. Rev. Biophys. Bioeng. 9:467 (1980), U.S. Pat. Nos. 4,235,871, 4,501,728, 4,837,028, and 5,019,369, incorporated herein by reference.

A liposome suspension containing a composition of the invention may be administered intravenously, locally, topically, etc. in a dose which varies according to, inter alia, the manner of administration, the composition of the invention being delivered, and the stage of the disease being treated.

For solid compositions, conventional nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more compositions of the invention of the invention, and more preferably at a concentration of 25%-75%.

For aerosol administration, the compositions of the invention are preferably supplied in finely divided form along with a surfactant and propellant. Typical percentages of compositions of the invention are 0.01%-20% by weight, preferably 1%-10%. The surfactant must, of course, be nontoxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% by weight of the composition, preferably 0.25-5%. The balance of the composition is ordinarily propellant. A carrier can also be included, as desired, as with, e.g., lecithin for intranasal delivery.

The constructs of the invention can additionally be delivered in a depot-type system, an encapsulated form, or an implant by techniques well-known in the art. Similarly, the constructs can be delivered via a pump to a tissue of interest.

In some embodiments of the invention, the compositions of the invention are administered ex vivo to cells or tissues explanted from a patient, then returned to the patient. Examples of ex vivo administration of gene therapy constructs include Arteaga, et al. Cancer Research 56(5):1098-1103 (1996); Nolta, et al. Proc Natl. Acad. Sci. USA 93(6):2414-9 (1996); Koc, et al. Seminars in Oncology 23 (1):46-65 (1996); Raper, et al. Annals of Surgery 223(2):116-26 (1996); Dalesandro, et al. J. Thorac. Cardi. Surg. 11(2):416-22 (1996); and Makarov, et al. Proc. Natl. Acad. Sci. USA 93(1):402-6 (1996).

The following examples are included for illustrative purposes and should not be considered to limit the present invention.

### EXAMPLES

The following examples are illustrative of particular embodiments of the invention. These examples should not be considered as limiting the scope of the invention as set forth above. Murine encephalomyocarditis (EMCV) was from ATCC, Gaithersburg, Md. Polyclonal antisera to human interferon cc was from Endogen (Woburn, Mass.). The neutralizing antibody for human interferon was obtained from PBL.

### 1. Effective Interferon-□ on Prostate Cancer Cell Proliferation

Three different prostate carcinoma cells, LNCaP (androgen dependent, ATCC #CRL 1740), PC-3 cells (androgen independent, ATCC #CRL 1435), and DU-145 (androgen independent, ATCC #HTB 81) were studied. The cells were grown in 5 different concentrations (10, 10², 10³, 10⁴, 10⁵ IU/ml) of interferon-α2b (Schering-Plough) for 72 hours in the following media: PC-3 was cultured in Ham's F12 K medium (GIBCO BRL) supplemented with 7% fetal bovine serum; DU-145 was cultured in DMEM (GIBCO BRL) supplemented with 10% fetal calf serum; LnCaP was cultured in RPMI 1640 (GIBCO BRL ) supplemented with 5% fetal bovine serum.

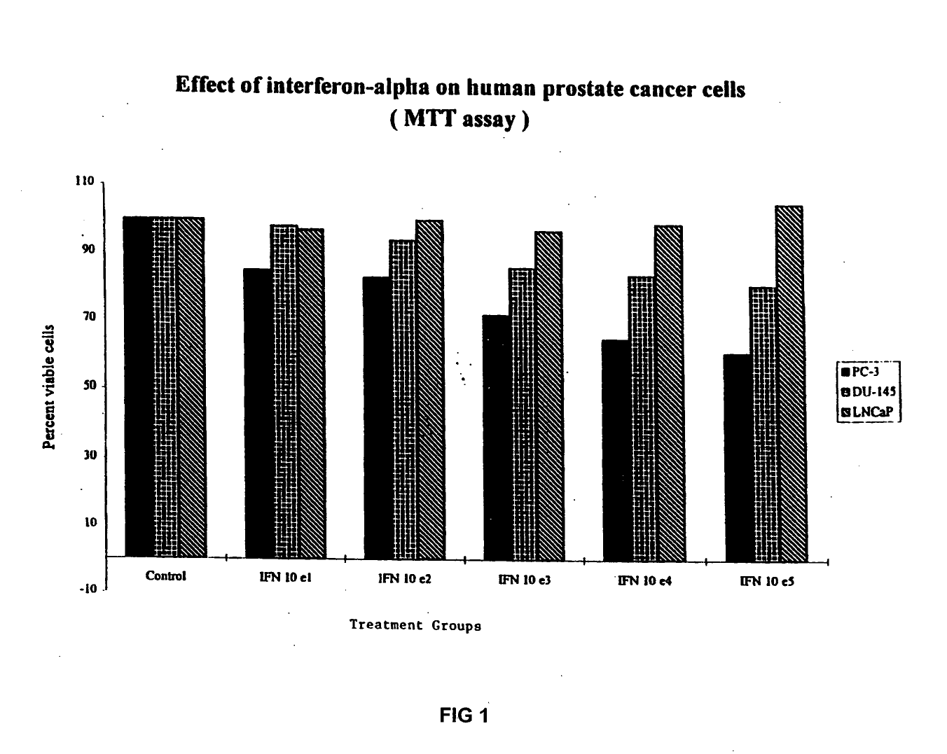

Antiproliferative effects of interferon were measured by MTT assay (Mossman J. Immunol. Methods 65:55 (1983)). PC-3 and DU-145 cells showed a consistent sensitivity to increasing concentrations of interferon plateauing at 10⁴ IU/ml. Androgen sensitive LNCaP cells did not respond to IFN. Between the two androgen refractory cells, PC-3 appeared more sensitive than DU-145 cells. These data are summarized in FIG. 1. Solid bars represent cell line PC-3 (androgen independent); cross-hatched bars represent cell line DU-145 (androgen independent); diagonal bars represent LNCaP (androgen dependent).

### Example 2. Construction of an Expression Cassette

An expression vector was constructed having the complete cDNA sequence of interferon α2b (IFN-α2b) and the complete signal sequence for IFN-α2b (Sreuli, et al. Science 209:1343-1347 (1980); Goeddel, et al. Nature 290:20-26 (1981); Henco, et al. J. Mol. Biol. 185:227-260 (1985)) under control of an approximately 600 bp basal promoter for the prostate specific antigen gene (PSA) for tissue specific expression of IFN-α in prostate carcinoma cells. Basically, full- length IFN-α2b cDNA having its putative signal leader at the 5′ end was cloned into the polycloning site at HindII and Eco RI downstream of the CMV promoter in the mammalian expression vector PCDNA3 (Invitrogen) to create plasmid DIFN. The 5′ flanking sequence of the PSA gene containing the PSA promoter (BBRC 161:1151-1159 (1989); Genebank #M27274) was inserted into the vector, replacing the CMV promoter, to create plasmid PSADIFN.

### Example 3. Cloning of Basal Promoters for Liver Specific Genes

5′ flanking sequences, including basal promoters, from four human liver specific genes, albumin (HAL), al-antitrypsin (HAT), a feto protein (AFP), and hydroxy-methyl-glutaryl CoA reductase (HMG), were subcloned from ATCC 65731, ATCC 61597, ATCC 65735, and ATCC 59567, respectively, into pCRScript vector for use in interferon gene delivery and its tissue specific expression in hepatic cells (Luskey Mol. Cell. Biol. 7(5):1881-1893 (1987); Minghetti, et al. J. Biol. Chem. 261(15):6747-6757 (1986); Long, et al. Biochemistry 23:4828-4837 (1984); Gibbs Biochemistry 26:1332-1343 (1987)).

After restriction enzyme mapping of the inserts in the pCRScript vector containing 5′-flanking sequences of the above genes for liver specific enzymes, the inserts were placed upstream of luciferase gene in the reporter plasmid, pGL3 (Promega) from pCRScript vector. Chinese hamster ovary (CHO-K1, ATCC # CCL-61), human hepatoma (HepG2, ATCC #NB 8065) and human hepatoma (Hep3B ATCC # HB 8064) cells were transfected by electroporation with these four constructs as well as by the control plasmid pGLC (PROMEGA Corp). pGLC contains the luciferase gene under the control of the SV40 promoter and the SV40 enhancer.

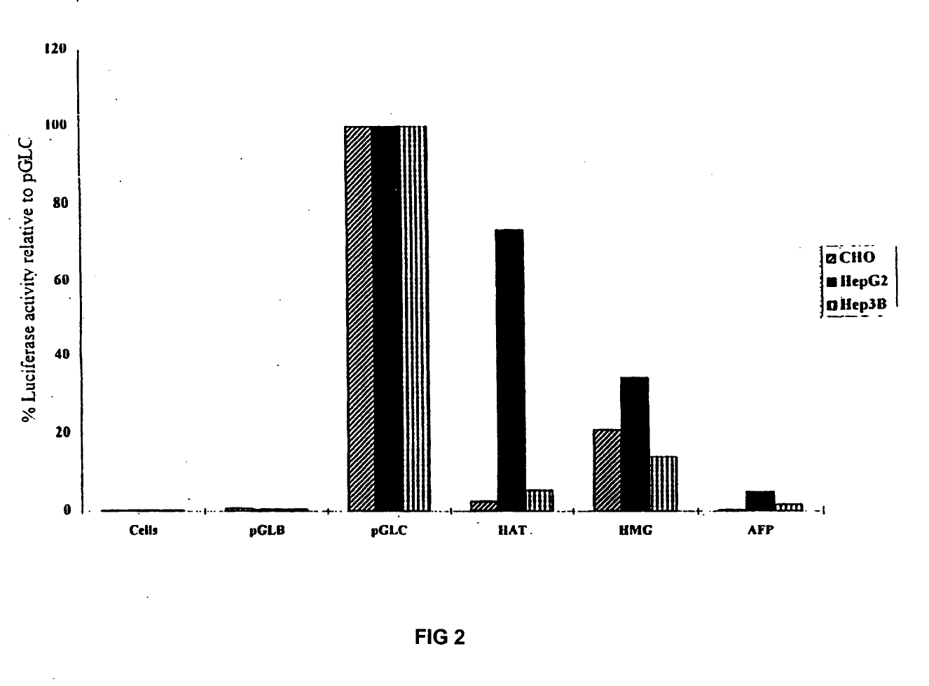

Luciferase expression by the four liver specific promoter sequences in the transfected cells was compared with the control plasmid pGLC and the vector pGL3. The data are summarized in FIG. 2 (HAL result not shown). Three cell types were transfected by DNA constructs having the salient features indicated. Solid bars represent human hepatoma HepG2 cells; cross-hatched bars represent Chinese hamster ovary cells (CHO); diagonal bars represent human hepatoma Hep3B cells (Hep3B). pGLB is a negative control plasmid; pGLC is a positive control plasmid; HAT denotes the human α1-antitrypsin promoter; HMG denotes the human hydroxy-methyl-glutaryl CoA reductase promoter; AFP denotes the human α-feto protein promoter. Of the four tissue-specific promoters, the human α1-antitrypsin (HAT) promoter appeared to be the best candidate for liver-specific expression under these conditions.

Expression driven by the HAT promoter can be further optimized by constructing an expression vector with a liver specific enhancer sequence such as the human α-fetoprotein enhancer (Watanabe, et al. J. Biol. Chem. 262(10):4812-4818 (1987), Genebank # J02693), the human albumin enhancer (Hayashi, et al. J. Biol. Chem. 267(21):14580-14585 (1992), Genebank # M92816), the human a-l microglobulin/bikunin enhancer (Rouet, et al. J. Biol. Chem. 267(29):20765-20773 (1992), Genebank # X67082), or the hepatitis B enhancer (Valenzuela, et al. Animal Virus Genetics ed. B. Biels, et al., p.p. 57-70, Academic Press, N.Y. (1981); Galibert, et al. Nature 281:646-650 (1979)).

### Example 4. Construction of Recombinant Adenoviruses Expressing IFN Species

Recombinant replication-deficient adenoviral vectors for the expression of interferon species were constructed by using standard procedures in substantial accordance with the teaching of Graham, F. L. and Prevec, L. (1995) Molecular Biotechnology Volume 3 Pages 207-220. A derivative of human adenovirus serotype 5, as described in Ahmed, et al. (1999, Human Gene Therapy 10:77-84) was used as the source of viral DNA backbone. The adenoviral vector backbone used for construction of the viruses possess deletions of the El region, protein IX, E3 and a partial E4 deletion as described in Wills, et al. (1994) Human Gene Therapy 5:1079-1088. Deletion of the E1 region renders the viruses replication-deficient, restricting their propagation to 293 cells which supply the Ad5 E1 gene products in trans (Graham, et al. (1977) J. Gen. Virol. 36:59-74.

The recombinant adenoviruses were constructed so as to direct the expression of a secreted and non-secreted form of interferon-α under the control of the human AFP promoter. The AFP promoter is well characterized in the art. An adenovirus transfer plasmid, pAAN, which contains the human AFP promoter was used to insert the coding sequence for human interferon α2b or α2α1. Recombinant adenoviruses were obtained by co-transfecting the linearized transfer plasmid and Cla I digested large fragment of viral DNA. Viral plaques were isolated and purified by column chromatography (Huyghe, et al., (1995) Human Gene Therapy 6:1403-1416, Shabram, et al. U.S. Pat. No. 5,837,520 issued Nov. 17, 1998). A control plasmid was constructed which contained no coding sequence following the promoter for use in constructing the control virus (rAdx). Secretion of the interferon was achieved by insertion of a 5′ DNA sequence encoding the naturally occurring human interferon-α2b signal peptide. The AFP promoter driven viruses encoding secretory interferons (α2b and α2α1 were designated as rAdIFNα2b and rAdIFNα2α1

The vectors expressing intracellular interferon were constructed by carrying out a polymerase chain reaction with the following primers. For the sense primer, a sequence corresponding to the initiating methionine and the sequence starting from the 24^{th} amino acid was used. This removes the secretory signal from this polypeptide. For the antisense primer the sequence at the 3′-end of the interferon was used. The vectors expressing the non-secreted interferon species were designated as rAdNSIα2b and rAdNSIα2α1. A control virus constructed as above however without the interferon-a species designated rAdx. The viruses were characterized by restriction enzyme digestion followed by DNA sequencing across the transgene and the promoter sequences. An additional control virus encoding CMV promoter driven β-galactosidase (rAd-β-gal) has been described previously (Smith, et al., (1997) Circulation 96:1899-1905.

The foregoing recombinant adenoviruses are deleted in early region 1, which makes them replication-deficient and restricts their propagation to cell lines capable of complementing these functions. Human embryonic kidney cell line 293 which supply the Ad5 E1 gene products in trans (Graham, et al., 1977) and was used for the propagation of recombinant adenoviruses described herein. 293 cells were grown in Dulbecco's modified Eagle's medium containing 10% bovine calf serum. The viruses were characterized by both restriction enzyme digestion and DNA sequencing across the transgene and the promoter sequences.

### Example 5: Detection of Interferon Protein

HepG2 cells (2.5×105) were seeded in a 6-well dish. HepG2 were grown in Dulbecco's modified Eagle's medium containing 10% fetal bovine serum. After overnight incubation, the cells were infected with 3×10⁸ particles/ml of the adenovirus constructs and allowed to grow for 2 days. Following this procedure, supernatants were collected. The cells were washed with PBS and harvested in lysis buffer [50 mM Tris.HCl pH 7.5, 250 mM NaCl, 0.1% NP-40, 50 mM NaF, 5 mM EDTA, 10 μg/ml aprotinin, 10 μg/ml leupeptin, 10 μg/ml phenylmethylsulfonylflouride]. Protein concentration was determined using a Bradford assay kit (commercially available from Bio-Rad). 10 μg of protein from each sample was loaded on 18% Tris-glycine-SDS-polyacrylamide gels and electrophoresed. The protein was transfered electrophoretically to Immobilon membrane (commercially available from Millipore) and probed with a polyclonal antisera to human interferon cc raised in sheep (commercially available from Endogen, Woburn, Mass.). A horseradish peroxidase-conjugated donkey-anti-sheep antibody (commercially available from Jackson Immunochemicals) was used as a secondary antibody and detection was carried out by chemiluminescence.

### Example 6: Quantitation of Interferon by Immunoassay

Aliquots from supernatants or cell extracts of HepG2 cells infected with different recombinant adenoviruses as prepared in accordance with the teaching of Example 4 above were analyzed using the ORIGEN® electrochemiluminescence detection system ((commercially available from Igen, Inc. Gaithersburg, Md.) and more fully described in Obenauer-Kutner, et al., (1997) J. Immunol. Methods 206:25-33. The assay was performed in substantial accordance with the instructions provided by the manufacturer.

### Example 7: Antiviral Assay

HepG2 cells were infected with the different viruses indicated. After 48 hr of infection, supernatants or cell extracts were collected for determination of antiviral activity by inhibition of cytopathic effect (CPE). A549 cells were plated in 96-well microtiter plate at a density of 4.7×10⁴ /cm² in 0.1 ml of complete media. Various dilutions of standard (Intron A® commercially available from Schering Corporation, Kenilworth N.J.) or test material in 50 μl were added to designated wells and incubated for 4 hours. Diluted murine encephalomyocarditis virus (EMCV, available from the ATCC) in 50 μl was added and incubated for 44 hours. Cell viability was measured using the CellTiter colorimetric assay (commercially available from Promega, Madison, Wis. Values for antiviral activity (I.U./ml) were determined by interpolation from an Intron A standard curve.

### Example 8: Measurement of Cell Proliferation

Cells (5×10³) were plated in a 96-well microtiter plate and allowed to grow overnight. They were infected with recombinant adenoviruses at particle concentrations indicated and allowed to grow for an additional 45 hours. 0.5 μCi of [³H]-thymidine (commercially available from Amersham) was added and incubated for three more hours. Cells were harvested on glass fiber filters and radioactivity was measured in a scintillation counter. [³H]-thymidine incorporation was expressed as % mean (+/−S.D.) of media control.

Although the foregoing invention has been described in some detail by way of illustration and example for purposes of clarity of understanding, it will be obvious that certain changes and modification may be practiced within the scope of the appended claims.

All references cited herein are incorporated by reference in their entirety for all purposes.

We claim:

1. A method for increasing a subject's resistance to viral infection, comprising administering to a selected tissue of said subject a recombinant adenoviral vector comprising a nucleic acid segment encoding an interferon α2b or an interferon α2α1 polypeptide, wherein said nucleic acid segment encoding the interferon-α polypeptide lacks a secretion leader sequence and wherein said nucleic acid segment is operatively linked to a promoter specific for the cells of said selected tissue, wherein the interferon alpha polypeptide is expressed in said cells.

2. The method of claim 1, wherein the interferon α polypeptide is interferon α2b.

3. The method of claim 1, wherein the interferon α polypeptide is interferon α2α1.

4. The method of claim 2 or 3, wherein the tissue comprises a liver cancer cell and wherein the promoter having specificity for the tissue of interest is a liver-specific promoter.

5. The method of claim 4, wherein the liver-specific promoter is the AFP promoter.

6. The method of claim 5, wherein the adenoviral vector is replication deficient.

7. The method of claim 4 wherein the vector is administered to the subject's tissue via the intrahepatic artery.

8. The method of claim 6, wherein the subject is diagnosed with hepatitis.

9. The method of claim 6, wherein the subject is diagnosed as at-risk for hepatitis.

10. The method of claim 2, wherein the vector is rAdNSIα2b.

11. The method of claim 3, wherein the vector is rAdNSIα2α1.
